(19) United States
(12) Patent Application Publication
(10) Pub. No.: US 2003/0157133 A1
(43) Pub. Date: Aug. 21, 2003

(54) Lipoteichoic acid immunogenic compositions and methods of making and using thereof

(76) Inventor: Joseph J. Drabick, Silver Spring, MD (US)

Correspondence Address: ATTN: MCMR-JA (Ms. Elizabeth Arwine), Office of the Staff Judge Advocate, U.S. Army Medical Research and Materiel Command, 504 Scott Street, Fort Detrick, MD, 21702-5012, US

(21) Appl. No.: 10/370,596

(22) Filed: Feb. 24, 2003

Related U.S. Application Data
(63) Continuation of application No. 09/948,553, filed on Sep. 10, 2001.
(60) Provisional application No. 60/231,959, filed on Sep. 12, 2000.

(51) Int. Cl. A61K 39/02; A61K 31/739

(52) U.S. Cl. 424/234.1; 514/54

(57) ABSTRACT

Compositions, vaccines, methods, and kits for treating, preventing, or inhibiting an infection or disease caused by a gram-positive organism are disclosed. The compositions comprise lipoteichoic acid from at least one gram-positive organism. Also disclosed are antibodies which specifically bind to lipoteichoic acid.

### CROSS REFERENCE TO RELATED APPLICATIONS

This application claims the benefit of U.S. Provisional Patent Application No. 60/231,959, filed Sep. 12, 2000, naming Joseph J. Drabick and David L. Hoover as co-inventors, which is herein incorporated by reference.

### ACKNOWLEDGMENT OF GOVERNMENT INTEREST

This invention was made by employees of the United States Army. The government has rights in the invention.

### BACKGROUND OF THE INVENTION

1. Field of the Invention

The invention relates to lipoteichoic acid and immunogenic compositions comprising lipoteichoic acid and methods of making and using thereof. In particular, the invention relates to a method of using lipoteichoic acid for treating, preventing or inhibiting gram-positive infections and diseases such as those caused by group A Streptococci.

2. Description of the Related Art

Group A streptococcal disease is of worldwide concern. Group A streptococci have been shown to be an important pathogen capable of existing both in a carrier state in an asymptomatic individual and in a symptomatic individual with symptoms of disease ranging from a mild sore throat, tonsillitis, or impetigo. If untreated these streptococcal infections could lead to glomerulonephritis, rheumatic fever, and permanent rheumatic heart disease and may also result in significant morbidity and mortality by causing valvular heart disease and renal failure. The virulence of streptococci and the devastating suppurative complications can rapidly cause death in otherwise healthy people. Other streptococcal infections and diseases such as streptococci pharyngitis, may lead to economic loss and epidemics.

By using antimicrobial agents specifically penicillin derived antibiotics, the causative organism can be readily eliminated following the prescribed regimen of appropriate antibiotic therapy. However, antibiotic treatments do not prevent one from obtaining a streptococcal infection or disease. Since an infected individual may be contagious and pass group A streptococcal organisms to others for up to about 72 hours after antimicrobial therapy has been initiated, antibiotic treatments are ineffective for providing protection against epidemics.

In group A streptococci and many other gram-positive bacteria, cell wall components include lipoteichoic acid (LTA). Since the 1970's LTA has been known to mediate the adherence of streptococci to epithelial cells. See Beachey, et al. (1976) J. Exp. Med. 143:759-771. LTA comprises a polymer chain of polyglycerophosphate as a backbone structure and glycolipids of cytoplasmic membrane origin. See Wicken et al. (1977) Biological Properties of Lipoteichoic Acids, Microbiology, pp. 360-365. Electronmicroscopy reveals that one end of LTA is linked to cytoplasmic membrane glycolipid while the other end extends to the cell outer surface of the bacteria through the cell wall peptidoglycan layer.

However, most research related to lipoteichoic acids has focused on preventing adherence of streptococci to host epithelial cells using LTA itself as a blocker. See Dale et al. (1994) J. Infect. Dis. 169:319-323; and Courtney et al. (1992) Microb. Pathog. 12(3):199-208. Other treatments against gram-positive bacterial infections and diseases are directed towards clearing LTA from the host by using macrophage scavenger receptor proteins. See U.S. Pat. No. 5,624,904. These methods, however, do not provide vaccines for inducing the production of opsonic antibodies against gram-positive bacteria or antibodies that inhibit the adherence of these organisms to host tissues.

Most vaccine research and development for streptococcal infections and diseases has focused on the M proteins, a heterogeneous group of structural proteins associated with virulence. See Fox (1964) J. Immunol. 93:826-837. An M protein is a fibrillar surface molecule which enables streptococcus to resist phagocytosis by macrophages and polymorpnonuclear neutrophiles of the infected host.

Unfortunately, within these M proteins are amino acid sequences shared with the heart muscle that results in cross-reactive antibodies that are responsible for rheumatic fever and vaccines with partially purified streptococcal M proteins often result in strong local and systemic reactions. See Schmidt (1969) J. Infect. Dis. 106:250-255; Potter et al., (1962) J. Clin. Invest. 41:301-310; Fox et al., (1969) J. Infect. Dis. 120:598-604; Fox et al., (1966) J. Exp. Med. 124:1135-1151; and Beachey et al., in Symposium on Bacterial Vaccine, Ed. J. B. Robbins, J. C. Hill, Brian Decker Publisher, New York, pp. 401-410 (1981).

Therefore, a need exists for a method for treating, preventing or inhibiting gram-positive infections and diseases such as those caused by group A Streptococci which does not induce complications due to cross-reactive antibodies.

### SUMMARY OF THE INVENTION

The present invention generally relates to compositions and methods for treating, preventing, or inhibiting infections and diseases caused by gram-positive organisms.

In some embodiments, the present invention relates to a pharmaceutical composition for treating, preventing, or inhibiting an infection or disease caused by a gram-positive organism which comprises lipoteichoic acid and a pharmaceutically acceptable carrier. The lipoteichoic acid is from at least one gram-positive organism which may belong to Streptococcus, Micrococcus, Lactobacillus, Staphylococcus, Bacillus, or Listeria. Preferably, the gram-positive organism is S. aureus, S. epidermidis, S. pyogenes, N. cereus, L. monocytogenes, or belongs to groups A, B, C, or G of Streptococcus. In some preferred embodiments, the gram-positive organism belongs to group A Streptococcus. Infection and diseases include septicemia, septic shock, toxic shock syndrome, multiple organ failure, infections due to medical devices, osteomyelitis, cellulitis, pharyngitis, wound infections, pneumonia, gastroenteritis, conjunctivitis, endocarditis, myositis, necrotizing fasciitis, bronchitis, septic arthritis, septic bursitis, neonatal sepsis, bacteremia, abscesses, suppurative phlebitis, sialoadenitis, dental caries, meningitis, and sinusitis.

In some embodiments, the invention relates to a pharmaceutical composition for treating, preventing, or inhibiting an infection or disease caused by a gram-positive organism comprising an antibody which specifically binds to a lipoteichoic acid and a pharmaceutically acceptable carrier. The lipoteichoic acid is from at least one gram-positive organism which may belong to Streptococcus, Micrococcus, Lactobacilltis, Staphylococcus, Bacillus, or Listeria. Preferably, the grain-positive organism is S. aureus, S. epidermidis, S. pyogenes, N. cereus, L. monocytogenes, or belongs to groups A, B, C, or G of Streptococcus. In some preferred embodiments, the gram-positive organism belongs to group A Streptococcus. Infection and diseases include septicemia, septic shock, toxic shock syndrome, multiple organ failure, infections due to medical devices, osteomyelitis, cellulitis, pharyngitis, wound infections, pneumonia, gastroentetitis, conjunctivitis, endocarditis, myositis, necrotizing fasciitis, bronchitis, septic arthritis, septic bursitis, neonatal sepsis, bacteremia, abscesses, suppurative phlebitis, sialoadenitis, dental caries, meningitis, and sinusitis.

In other embodiments, the invention relates to a vaccine for providing protection against an infection or a disease caused by a gram-positive organism comprising a lipoteichoic acid or an immunogenic composition comprising lipoteichoic acid. The lipoteichoic acid is from at least one gram-positive organism which may belong to Streptococcus, Micrococcus, Lactobacillus, Staphylococcus, Bacillus, or Listeria. Preferably, the gram-positive organism is S. aureus, S. epidermidis, S. pyogenes, N. cereus, L. monocytogenes, or belongs to groups A, B, C, or G of Streptococcus. In additional preferred embodiments, the gram-positive organism belongs to group A Streptococcus. The vaccine may further comprise a suitable adjuvant. Infection and diseases include septicemia, septic shock, toxic shock syndrome, multiple organ failure, infections due to medical devices, osteomyelitis, cellulitis, pharyngitis, wound infections, pneumonia, gastroenteritis, conjunctivitis, endocarditis, myositis, necrotizing fasciitis, bronchitis, septic arthritis, septic bursitis, neonatal sepsis, bacteremia, abscesses, suppurative phlebitis, sialoadenitis, dental caries, meningitis, and sinusitis.

The invention further relates to a method of treating, preventing, or inhibiting an infection or disease caused by a gram-positive organism in a subject comprising administering to the subject lipoteichoic acid, a pharmaceutical composition or a vaccine comprising lipoteichoic acid, antibodies which specifically bind to lipoteichoic acid, or a combination thereof. Infection and diseases include septicemia, septic shock, toxic shock syndrome, multiple organ failure, infections due to medical devices, osteomyelitis, cellulitis, pharyngitis, wound infections, pneumonia, gastroenteritis, conjunctivitis, endocarditis, myositis, necrotizing fasciitis, bronchitis, septic arthritis, septic bursitis, neonatal sepsis, bacteremia, abscesses, suppurative phlebitis, sialoadenitis, dental caries, meningitis, and sinusitis.

In additional embodiments, the invention relates to a method of immunizing a subject against an infection or disease caused by a gram-positive organism comprising administering to the subject an immunogenic amount of lipoteichoic acid. The immunogenic amount is an amount that induces protective anti-adherence, opsonophagocytic antibodies against a gram-positive organism, or both in the subject. Infection and diseases include septicemia, septic shock, toxic shock syndrome, multiple organ failure, infections due to medical devices, osteomyelitis, cellulitis, pharyngitis, wound infections, pneumonia, gastroenteritis, conjunctivitis, endocarditis, myositis, necrotizing fascuitis, bronchitis, septic arthritis, septic bursitis, neonatal sepsis, bacteremia, abscesses, suppurative phlebitis, sialoadenitis, dental caries, meningitis, and sinusitis.

In the embodiments of the invention, the subject is a mammal, preferably human. In some embodiments, the invention relates to a kit for treating, preventing, or inhibiting an infection or disease caused by a gram-positive organism in a subject comprising a composition a therapeutically effective amount of a lipoteichoic acid. The therapeutically effective amount is an amount that induces protective anti-adherence, opsonophagocytic antibodies against a gram-positive organism, or both in the subject. The kit may further comprise a device for administering the composition, directions for use, reagents for detecting or measuring the protective anti-adherence, opsonophagocytic antibodies, or both in the subject, or a combination thereof. Infection and diseases include septicemia, septic shock, toxic shock syndrome, multiple organ failure, infections due to medical devices, osteomyelitis, cellulitis, pharyngitis, wound infections, pneumonia, gastroenteritis, conjunctivitis, endocarditis, myositis, necrotizing fasciitis, bronchitis, septic arthritis, septic bursitis, neonatal sepsis, bacteremia, abscesses, suppurative phlebitis, sialoadenitis, dental caries, meningitis, and sinusitis.

In some embodiments, the invention relates to a kit for treating, preventing, or inhibiting an infection or disease caused by a gram-positive organism in a subject comprising a composition comprising at least one antibody which specifically binds to a lipoteichoic acid. The kit may further comprise further comprising directions for use, reagents for detecting or measuring the amount of gram-positive organisms in the subject, or both. Infection and diseases include septicemia, septic shock, toxic shock syndrome, multiple organ failure, infections due to medical devices, osteomyelitis, cellulitis, pharyngitis, wound infections, pneumonia, gastroenteritis, conjunctivitis, endocarditis, myositis, necrotizing fasciitis, bronchitis, septic arthritis, septic bursitis, neonatal sepsis, bacteremia, abscesses, suppurative phlebitis, sialoadenitis, dental caries, meningitis, and sinusitis.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention and together with the description serve to explain the principles of the invention.

### DESCRIPTION OF THE DRAWINGS

This invention is further understood by reference to the drawings wherein:

FIG. 1 is an immunofluorescence of group A Streptococci with LTA monoclonal antibodies.

### DETAILED DESCRIPTION OF THE INVENTION

Generally, the present invention provides a method of using lipoteichoic acid (LTA) for treating, preventing or inhibiting gram-positive infections and diseases such as those caused by group A Streptococci. The present invention also provides an immunogenic composition comprising lipoteichoic acid (LTA) and methods of making and using thereof.

LTA or an immunogenic composition comprising LTA may be used to raise antibodies useful for prophylactic and diagnostic purposes. Diagnostics are particularly useful in monitoring and detecting various gram-positive infections and diseases such as those caused by group A Streptococci. Another embodiment of the invention uses the immunogenic composition as an immunogen for use in active, passive, or both immunogenic protection of a subject against gram-positive infections and diseases such as those caused by group A Streptococci.

For example, LTA has been implicated in gram-positive shock and multiple organ failure. See Kengatharan, et al. (1998) J. Exp. Med. 188(2):305-318, DeKimpe et al. (1995) PNAS USA 92:10359-10363, and Wang et al. (2000) Infect. and Immun. 68(7):3965-3970, which are herein incorporated by reference. Therefore, LTA or an immunogenic composition comprising LTA may be used in methods for treating, preventing, or inhibiting gram-positive diseases or infections. Gram-positive infections and diseases refer to those caused by or related to gram-positive bacteria. Gram-positive diseases and infections include septicemia, septic shock, toxic shock syndrome, multiple organ failure, infections relating to medical devices, such as prosthetics and catheters, osteomyelitis, cellulitis, pharyngitis, wound infections, pneumonia, gastroenteritis, conjunctivitis, endocarditis, myositis, necrotizing fasciitis, bronchitis, septic arthritis, septic bursitis, neonatal sepsis, bacteremia, abscesses, suppurative phlebitis, sialoadenitis, dental caries, meningitis, and sinusitis.

Examples of gram-positive bacteria having LTA include those belonging to the genera such as Streptococcus, Micrococcus, Lactobacillus, Staphylococcus, Bacillus, and Listeria. The preparation of LTA from whole cells, or a cell envelope fraction of these bacteria may be obtained, for example, according to the method described by Beachey et al. (1979) Infect. Immun. 23:618-625, which is herein incorporated by reference.

LTA or an immunogenic composition comprising LTA may be used as or in a vaccine against a variety of gram-positive bacteria. The highly conserved nature of LTA obviates the need to use strain specific LTA. The use of LTA also obviates the problem of immunologic cross-reactivity with a subject's cardiac tissue. LTA may be used to induce the production of opsonophagocytic antibodies which mediate the destruction of a bacterium expressing LTA by host leukocytes. Since LTA is conserved between different group A streptococci, group B streptococci, and a variety of other gram-positive organisms, LTA may be used as a vaccine by inducing protective anti-adherence and opsonophagocytic antibodies against a wide variety of gram-positive organisms.

LTA may be used as a vaccine alone or LTA may be conjugated to a carrier protein or incorporated in liposomes by standard techniques known in the art. LTA or an immunogenic composition comprising LTA may be administered parenterally or intranasally to a subject to induce mucosal immunity.

Since LTA is amphipathic, it is soluble in both water and lipophilic medium and may be formulated by a conventional formulation process into any desired form. The LTA or the immunogenic composition comprising LTA may be used alone or in combination with a suitable adjuvant such as alum, aluminum hydroxide, or aluminum phosphate. The LTA or the immunogenic composition may be conjugated to other suitable immunogenic agents by standard methods known in the art. Suitable immunogenic agents include outer membrane protein of Neisseria meningitidis, diphtheria toxoid, tetanus toxoid, and the like.

The LTA immunogen is typically used to prepare antibodies by immunizing a suitable subject, such as a rabbit, goat, mouse or other mammal with the immunogen. An appropriate immunogenic preparation may include an adjuvant, such as Freund's complete or incomplete adjuvant, or similar immunostimulatory agent. Immunization of a suitable subject with the immunogenic preparation induces a polyclonal anti-LTA antibody response.

Accordingly, another aspect of the invention pertains to anti-LTA antibodies and fragments thereof The term “antibody” as used herein refers to immunoglobulin molecules and fragments thereof of immunologically active portions of immunoglobulin molecules, i.e., molecules that contain an antigen binding site which specifically binds an antigen, such as LTA. Examples of immunologically active portions of immunoglobulin molecules include F(ab) and F(ab′)₂ fragments which can be generated by treating the antibody with an enzyme such as pepsin.

The invention provides polyclonal and monoclonal antibodies that bind LTA. The term “monoclonal antibody” or “monoclonal antibody composition”, as used herein, refers to a population of antibody molecules that contain only one species of an antigen binding site capable of immunoreacting with a particular epitope of LTA. A monoclonal antibody composition thus typically displays a single binding affinity for a particular LTA.

Polyclonal anti-LTA antibodies can be prepared as described above by immunizing a suitable subject with LTA or an immunogenic composition comprising LTA. The anti-LTA antibody titer in the immunized subject can be monitored over time by standard techniques, such as with an enzyme linked immunosorbent assay (ELISA). If desired, the antibody molecules directed against LTA can be isolated from the mammal and further purified by well known techniques, such as protein A chromatography to obtain the IgG fraction. At an appropriate time after Immunization, e.g., when the anti-LTA antibody titers are highest, antibody-producing cells can be obtained from the subject and used to prepare monoclonal antibodies and hybridomas by standard techniques known in the art. The antibodies may be humanized or completely human antibodies prepared by methods known in the art.

LTA or the immunogenic composition comprising LTA can be used as an immunogen to generate antibodies against gram-positive bacteria using standard techniques for polyclonal and monoclonal antibody preparation. Preferred epitopes encompassed by the immunogen are regions of LTA which play a role in epithelial cell adhesion.

The LTA or the immunogenic composition may be incorporated into pharmaceutical compositions suitable for administration. Such compositions typically comprise the LTA or the immunogenic composition and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Supplementary active compounds include other antigenic determinants against gram-positive bacteria such as M proteins, peptidoglycans, teichoic acids, other surface proteins, carbohydrates, and the like.

The pharmaceutical composition of the invention is formulated to be compatible with its intended route of administration. Examples of routes of administration include parenteral, intravenous, intradermal, subcutaneous, oral, transdermal, transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol, for example, glycerol, propylene glycol, and liquid polyetheylene glycol, and the like, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, adjuvant materials, or both can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories e.g., with conventional suppository bases such as cocoa butter and other glycerides or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 i. e., the concentration of the test compound which achieves a half-maximal inhibition of symptoms as determined in cell culture. Such information can be used to more accurately determine useful doses in humans. Levels in plasma may be measured, for example, by high performance liquid chromatography.

As defined herein, a therapeutically effective amount of LTA or the immunogenic composition of the invention ranges from about 0.0001 to about 0.1 mg/kg body weight, preferably about 0.0001 to about 0.01 mg/kg body weight, more preferably about 0.0001 to about 0.001 mg/kg body weight, and even more preferably about 0.0005 to about 0.001 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of the pharmaceutical composition can include a single treatment or, preferably, can include a series of treatments. It will also be appreciated that the therapeutically effective amount of the pharmaceutical composition used for treatment may increase or decrease over the course of a particular treatment. Changes in dosage may result and become apparent from the results of diagnostic assays as described herein.

Anti-LTA antibodies may be made by standard methods known in the art. For example, a murine monoclonal antibody against streptococcal LTA may be made from BALB/c mice hyperimmunized with streptococcal LTA. The LTA used may be obtained from commercial vendors such as Sigma (St. Louis, Mo.) or prepared by acid hydrolysis. Anti-LTA antibodies can be used to isolate LTA or bacteria having LTA by standard techniques, such as affinity chromatography or immunoprecipitation. Anti-LTA antibodies can facilitate the purification of natural LTA from cells. Moreover, anti-LTA antibodies can be used to detect LTA in a sample in order to evaluate the abundance and pattern of expression of the LTA. The anti-LTA antibodies may be used to passively transfer immunity against gram-positive organisms to individuals who lack active immunity. The anti-LTA antibodies may be used to inhibit the binding of gram-positive organisms to mammalian cells.

The antibodies can be used diagnostically to monitor LTA levels in tissue as part of a clinical testing procedure, e.g., to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, the antibody or fragment thereof may be conjugated to a therapeutic moiety such as a cytotoxin, a therapeutic agent or a radioactive metal ion. A cytotoxin or cytotoxic agent includes any agent that is detrimental to bacterial cells. Examples include antibiotics with activity against gram-positive organisms such as beta-lactams, macrolides, tetracyclines, penems, aminoglycosides, glycopeptides, and the like.

The pharmaceutical compositions can be included in a kit. The kit may comprise instructions for use, reagents for diagnostic assays, and devices for administration.

A gram-positive bacterial infection animal model may be used to study anti-LTA immune sera, anti-LTA monoclonal antibodies, and active LTA vaccines such as LTA-GBOMP to determine, for example, if passive administration of the anti-LTA antibody as immune serum from immunized animals or the anti-LTA monoclonal antibody could prevent death relative to a control antibody in the Dale model of group A Streptococcal infection. Alternatively, animal models for other gram-positive organisms such as Staphylococcus aureus and Staphyloccus epidermidis may be used.

The following examples are intended to illustrate but not to limit the invention.

### EXAMPLE 1

### Anti-LTA Monoclonal Antibodies

Four BALB/c mice were each immunized with 100 μg of LTA in Freund's Adjuvant, complete. About two weeks later, each mouse was boosted with 50 μg of LTA in Freund's Adjuvant, incomplete. Two weeks later, the mice were eye-bled and the serum was screened by an ELISA assay to establish LTA titers. The two mice having the highest titers were each boosted again with 50 μg of LTA in Freund's Adjuvant, incomplete.

After three days, the splenocytes of the injected mice were fused with the X63/Ag 8.653 myeloma cell line (ATCC #CRL-1580) using polyethylene glycol. Cloning was performed in Iscoves media with 10% fetal calf serum (Hazelton Bioproducts, Vienna, Va.) supplemented and selected with HAT (GIBCO, Long Island, N.Y.). The cells were plated and given fresh media and examined every three days for colony growth.

Colonies were screened for antibody production and positive cells were cloned several times by limiting dilution. Fully cloned cells were grown in T-175 culture flasks. The cells were injected i.p. into pristane primed BALB/c mice for ascites production. After two weeks the mice were tapped for ascites and the ascites were screened for anti-LTA antibody production.

### EXAMPLE 2

### ELISA for Anti-LTA Antibodies

96 well polystyrene ELISA plates were coated with 50 μg/cc of poly-1-lysine in PBS overnight. The next day the plates were washed and coated with 10 μg/cc of LTA in PBS and incubated overnight. The following day the plates were blocked with Casein and BSA blocking buffer for 2 hours.

Supernatants from the hybridomas were tested undiluted and with serial 1:2 dilutions across the plate. Ascites were tested beginning at 1:100 dilution with 1:2 dilutions across the plate. Samples were incubated 4 hours then washed 3 times.

The secondary antibody, phosphatase labeled goat anti-mouse IgG was diluted 1:300 in casein filler/buffer. The plates were incubated for 2 hours then washed 3 times. The phosphatase substrate (Sigma, St. Louis, Mo.) was dissolved in diethanolamine buffer and added to the plates for 30 minutes. The plates were read at 405 nm.

### EXAMPLE 3

### Agglutination Assay

Group A streptococci were grown in Todd-Hewitt broth (Difco, Detroit, Mich.) for 3 hours at 37° C. Other broth grown bacteria may be substituted. The bacteria were pelleted and washed with PBS three times. The pellet was resuspended in 1 mL of PBS and the optical density was read at 750 mn. The concentration of the bacteria was adjusted with PBS so that the optical density was equal to 0.5 at 750 nm which corresponds to about 2×10⁸ bacterium per mL.

10 fold dilutions of the LTA monoclonal antibody were made in PBS. The control was PBS without bacteria. 50 μL of each of the dilutions were added to a well of an agglutination slide. 50 μL of the bacterial suspension was added to each well. The samples were mixed well and rotated gently for 10 minutes. Then each well was examined for agglutination.

### EXAMPLE 4

### ELISA for Anti-LTA Antibodies Against Intact Whole Organisms

To determine whether an anti-LTA antibody can react with a whole organism that has LTA, the organism was cultured and then washed 3 times by pelleting and resuspending with PBS. The pellet was resuspended in 1 mL of PBS and the optical density was read at 750 nm and adjusted to with PBS to an optical density of 0.6 to correspond to about 1×10⁸ organisms per mL. 25 μL was placed in a soft U-bottom 96 well plates (Gibco, Long Island, N.Y.). The plate was spun at 400×g for 5 minutes at room temperature. 25 μL of 0.25% freshly prepared glutaraldehyde solution were added and incubated for 5 minutes. The plate was washed 3 times with PBS. The plates were blocked with filler for at least 4 hours and then washed. Each sample was examined for reactivity to LTA, Ricin, and Fran4. Fran4, an IgM Mab against Francisella tularensis lipopolysaccharide, and ricin, an IgM Mab against the plant lectin, ricin, were used as negative controls, i.e. monoclonal antibodies of the same class (IgM) against different antigens. The results are shown in Table 1.

TABLE 1
Reactivity of LTA Mab (Reactivity in O.D. units at 405 nm)

| | Mab | | |
|---|---|---|---|
| Organism | LTA | Ricin | Fran4 |
| Group A Strep | 1700 | 0 | 0 |
| Group B Strep | 76 | 0 | 0 |
| Staph aureus | 50 | 8 | 1 |
| Staph epi | 7600 | 40 | 5 |
| Bacillus cereus | 120 | 3 | 0 |
| Brucella melitensis | 1 | 2 | 0 |
| (negative control) | | | |

These results show that anti-LTA antibody can specifically react with whole organisms which have LTA while the negative control monoclonal antibodies, ricin and Fran4, do not. These results show that LTA in its natural state in the outer wall of gram-positive organisms can be recognized by anti-LTA antibodies which is a prerequisite for LTA to be able to react with antibodies if LTA is to be a protective antigen.

### EXAMPLE 5

### Opsonophagocytosis Assay

Since reliable animal models of group A streptococcal virulence to test the therapeutic efficacy of monoclonal antibodies against LTA or LTA or an immunogenic composition comprising LTA as an active vaccine do not exist, an in vitro opsonophagocytosis assay which correlates with the in vivo efficacy of antibodies against other bacterial organisms was used. This assay measures the ability of an antibody to work in concert with polymorphonuclear white cells to kill growing bacteria in vitro.

Bacteria were placed in trypticase soy broth to an optical density of about 0.3 to about 0.4 and incubated and shaken at 37° C. for about 2 to about 3 hours until a log phase or an optical density of about 1.0 to about 1.2 was reached. The bacteria were spun at 2200 rpm for 5 minutes to pellet. The pellet was washed with 10.0 mL normal saline, vortexed and repelleted at 2200 rpm for 5 minutes. All but the last 0.3 mL of normal saline was removed and then vortexed.

To an optical density tube containing 5.0 mL of normal saline, the vortexed bacteria were added dropwise until an optical density of 0.3 was obtained. The tube was placed on ice until further use.

A fresh blood sample was placed in a tube with an equal volume of 2.0% Dextran in saline with 0.2 mL of heparin. The tube was mixed by inversion. The cells were allowed to settle for about 30 to about 45 minutes. The supernatant was removed and placed in a new tube. A volume that was half the total volume of the supernatant of LSM was placed at the bottom of the tube without mixing with the supernatant. The tube was spun at 2200 rpm for 10 minutes. The supernatant was removed. Red blood cells remaining in the pellet were removed by treating with 6.0 mL of cold water and 2.0 mL of 3.5% sodium chloride and then mixed gently and spun for 10 minutes at 2200 rpm. The supernatant was removed and the pellet was resuspended in 5.0 mL of Hank's balanced salt solution. 20 μL of resuspended cells were placed in 380 μL of 3% acetic acid and trypan blue and mixed. A hemacytometer was used to count all four corners and then averaged. The concentration was adjusted after spining to 23 million per mL.

To a sterile 96 well plate, 60 μL of the blood cell sample, 20 μL of NHS, 10 μL of the bacterial sample, 10 μL of diluent (Hanks balanced salt solution) were added to each well. For each well, two sets of dilution tubes, 1.0 mL and 2.0 mL of 0.1% BSA in dH₂O, were set up for time zero and 120 minutes.

After the plate is loaded, time zero dilutions were started by taking 10 μL samples from the plate and adding each to the appropriate 2.0 dilution tube mix. Then 10 μL of 2.0 dilution tube mix was placed in the 1.0 mL dilution tube. The plate was covered and incubated at 37° C. for 2 hours on a microshaker. 10 μL of each dilution were plated, covered, dried, and incubated overnight at 37° C.

Table 2 shows the results of the opsonophagocytosis assay.

TABLE 2
Results of Opsonophagocytosis Assay Utilizing Group A Streptococci

| REAGENT | DILUTION | % KILLING |
|---|---|---|
| PBS | — | 0 |
| LTA ascites | 1/3000 | 99 |
| LTA ascites | 1/10000 | 23 |
| Ricin ascites* | 1/3000 | 0 |
| Ricin ascites* | 1/10000 | 0 |
| LTA supernatant | 1/1 | 80 |
| LTA supernatant | 1/10 | 0 |
| LTA immune serum | 1/1 | 73 |
| LTA inmmne serum | 1/10 | 50 |
| Normal mouse serum | 1/1 | 0 |

*ricin Mab is murine IgM directed against the plant lectin, ricin.

As illustrated by Table 2, LTA immune sera, anti-LTA monoclonal antibody culture media supernatant, and anti-LTA monoclonal ascites exhibit excellent opsonophagocytic function resulting in a high level killing of the organism relative to non-immune serum and a monoclonal antibody which does not recognize LTA.

### EXAMPLE 6

### Lipoteichoic Acid Immunofluorescence Antibody Staining

Gram-positive organisms, S. aureus, S. epidermidis, Streptococci groups A, B, C, and G. N. cereus, and L. monocytogenes were used. The negative control used was E. coli, a gram-negative organism. Each organism was cultured in Todd-Hewitt broth (Difco, Detroit, Mich.) for 3 hours at 37° C. Each organism was then transferred to a 15 mL centrifuge tube and washed three times with PBS by pelleting and resuspending. The pellet was resuspended in about 2 mL of PBS. A drop of each organism suspension was placed in a well of an immunofluorescence slide. Each slide was air dried and fixed with methanol for 5 minutes.

The slides were placed in a moist chamber. 30 μL of a LTA monoclonal antibody solution was placed in each well over each organism. The slides were covered and incubated for 30 minutes at 37° C. Each slide was washed gently by dipping each slide in a 50 mL centrifuge tube filled with PBS several times. The slides were drained and air dried and placed into the moist chamber. 30 μL of PITC conjugated goat anti-mouse IgM (Kirkgaard & Perry, Gaithersberg, Md.) in a 1:10 dilution was placed into each well. The slides were covered and incubated for 30 minutes at 37° C. The slides were washed gently in PBS, air dried and examined under a fluorescence microscope.

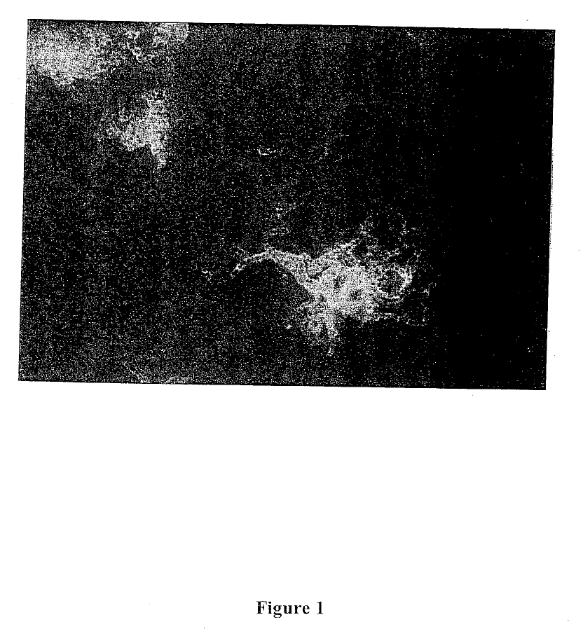

All organisms except E. coli were positive. FIG. 1 is an example of a positive immunofluorescence of group A Streptococci with LTA monoclonal antibodies.

EXAMPLE 7

### LTA Vaccine Conjugate

An active conjugate LTA vaccine can be made to increase the immunogenicity of LTA. To prepare a LTA-GBOMP non-covalent complex, 5.9 mg of LTA (Sigma, St. Louis, Mo.) was added to 2.0 mL sterile 0.9% NaCl and stirred for 5 minutes to give a hazy solution to which 5.0 mL of N. meningitides group B outer membrane protein (GBOMP) (1.398 mg/mL) in TWEEN 0.1% buffer.

The mixture was placed into a dialysis tubing (1000 M.W. cutoff) and placed for dialysis vs. 500 mL of 0.9% NaCl at 5° C. for 2 days without stirring. Dialysis was then continued with stirring at 5° C. for 2 days. The dialysis solution was changed to a fresh 500 mL 0.9% NaCl and dialysis continued at 5° C. for 3 days. The dialysis solution was changed one more time and dialysis continued for another 3 days.

The dialyzed LTA-GBOMP non-covalent complex was taken out and filtered with a 0.45 μm membrane. Analysis by the phenol-sulfuric acid method showed a presence of 320 μg LTA/mL and measurement of GBOMP by absorbance at 280 nm showed a presence of 527 μg GBOMP/mL. Therefore the ratio of GBOMP to LTA is 1.6:1.

The Dale model of group A Streptococcal infection may be used to test active immunization using the LTA-GBOMP non-covalent complex versus GBOMP alone to determine whether the LTA-GBOMP non-covalent complex could induce protective antibodies to LTA and survival to challenge with the virulent Streptococci.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A pharmaceutical composition for treating, preventing, or inhibiting an infection or disease caused by a gram-positive organism comprising a lipoteichoic acid and a pharmaceutically acceptable carrier.

2. The pharmaceutical composition of claim 1, wherein the lipoteichoic acid is from at least one gram-positive organism.

3. The pharmaceutical composition of claim 1, wherein the gram-positive organism belongs to Streptococcus, Micrococcus, Lactobacillus, Staphylococcus, Bacillus, or Listeria.

4. The pharmaceutical composition of claim 1, wherein the gram-positive organism is S. aureus, S. epidermidis, S. pyogenes, N. cereus, or L. monocytogenes.

5. The pharmaceutical composition of claim 1, wherein the gram-positive organism belongs to Streptococcus group A, B, C, or G.

6. The pharmaceutical composition of claim 1, wherein the gram-positive organism belongs to group A Streptococcus.

7. A pharmaceutical composition for treating, preventing, or inhibiting a infection or disease caused by a gram-positive organism comprising an antibody which specifically binds to a lipoteichoic acid and a pharmaceutically acceptable carrier.

8. The pharmaceutical composition of claim 7, wherein the lipoteichoic acid is from at least one gram-positive organism.

9. A vaccine for providing protection against an infection or a disease caused by a gram-positive organism comprising a lipoteichoic acid or an immunogenic composition comprising lipoteichoic acid.

10. The vaccine of claim 9, wherein the lipoteichoic acid is from at least one gram-positive organism.

11. The vaccine of claim 10, wherein the gram-positive organism belongs to Streptococcus, Micrococcus, Lactobacillus, Staphylococcus, Bacillus, or Listeria.

12. The vaccine of claim 11, wherein the gram-positive organism is S. aureus, S. epidermidis, S. pyogenes, N. cereus, or L. monocytogenes.

13. The vaccine of claim 12, wherein the gram-positive organism belongs to Streptococcus group A, B, C, or G.

14. The vaccine of claim 12, wherein the gram-positive organism belongs to group A Streptococcus.

15. The vaccine of claim 9, further comprising a suitable adjuvant.

16. A method of treating, preventing, or inhibiting an infection or disease caused by a gram-positive organism in a subject comprising administering to the subject a therapeutically effective amount of a pharmaceutical composition according to claim 1.

17. A method of treating, preventing, or inhibiting an infection or disease caused by a gram-positive organism in a subject comprising administering to the subject a therapeutically effective amount of a vaccine according to claim 7.

18. A method of treating, preventing, or inhibiting an infection or disease caused by a gram-positive organism in a subject comprising administering to the subject a therapeutically effective amount of a vaccine according to claim 9.

19. A method of immunizing a subject against an infection or disease caused by a gram-positive organism comprising administering to the subject an immunogenic amount of lipoteichoic acid.

20. The method of claim 19, wherein the immunogenic amount is an amount that induces protective anti-adherence, opsonophagocytic antibodies against a gram-positive organism, or both in the subject.

21. The method of claim 19, wherein the subject is a mammal.

22. The method of claim 21, wherein the mammal is human.

23. A kit for treating, preventing, or inhibiting an infection or disease caused by a gram-positive organism in a subject comprising a composition a therapeutically effective amount of a lipoteichoic acid.

24. The kit of claim 23, wherein the therapeutically effective amount is an amount that induces protective anti-adherence, opsonophagocytic antibodies against a gram-positive organism in the subject, or both.

25. The kit of claim 23, further comprising a device for administering the composition, directions for use, reagents for detecting or measuring the protective anti-adherence, opsonophagocytic antibodies, or both in the subject, or a combination thereof.

26. A kit for treating, preventing, or inhibiting an infection or disease caused by a gram-positive organism in a subject comprising a composition comprising at least one antibody which specifically binds to a lipoteichoic acid.

27. The kit of claim 26, further comprising directions for use, reagents for detecting or measuring the amount of gram-positive organisms in the subject, or both.

28. The method of claim 19, wherein the infection or disease is septicemia, septic shock, toxic shock syndrome, multiple organ failure, an infection due to a medical device, osteomyelitis, cellulitis, pharyngitis, a wound infection, pneumonia, gastroenteritis, conjunctivitis, endocarditis, myositis, necrotizing fasciitis, bronchitis, septic arthritis, septic bursitis, neonatal sepsis, bacteremia, an abscess, suppurative phlebitis, sialoadenitis, dental caries, meningitis, or sinusitis.
